(19) United States
(12) Plant Patent Application Publication
(10) Pub. No.: US 2015/0163982 P1
(43) Pub. Date: Jun. 11, 2015

(54) Bougainvillea plant named 'Vista2'

(50) ;

(71) Applicant: Jerome O'Neill, Juana Diaz, PR (US)

(72) Inventor: Jerome O'Neill, Juana Diaz, PR (US)

(21) Appl. No.: 13/998,814

(22) Filed: Dec. 9, 2013

(51) Int. Cl. A01H 5/00

(52) U.S. Cl. PLT/256

(57) ABSTRACT

A new and distinct Bougainvillea cultivar named ‘Vista2’ is disclosed, characterized by orange bracts. Plants are very compact. The new variety is a Bougainvillea, typically produced as an ornamental plant.

### LATIN NAME OF THE GENUS AND SPECIES

Bougainvillea communis

### VARIETY DENOMINATION

‘Vista2’

### BACKGROUND OF THE INVENTION

The new cultivar is the product of chance discovery. The new variety originated as naturally occurring whole plant mutation of the unpatented variety Bougainvillea communis ‘Flame’. The inventor discovered the variety in a commercial planting of the parent variety in Juana Diaz, Puerto Rico in August of 2012.

After identifying the interesting flower color mutation, the inventor observed the single plant mutation for several months. First propagation was performed by vegetative cuttings during October 2012, in Juana Diaz, Puerto Rico. Access to all plants was restricted, as plants were kept in a facility belonging to the inventor and not open to the public.

Through subsequent propagation by vegetative cuttings, multiple generations have been reproduced, which have shown that the unique features of this cultivar are stable and reproduced true to type.

### SUMMARY OF THE INVENTION

The cultivar ‘Vista2’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Vista2’. These characteristics in combination distinguish ‘Vista2’ as a new and distinct Bougainvillea cultivar:
1. Very compact growth habit
2. Color of mature bract is orange

### PARENT COMPARISON

Plants of the new cultivar ‘Vista2’ are similar to the parent, in most horticultural characteristics. The new variety, however, produces bracts that are orange in color, whereas bracts of the parent variety are an orange-red.

### COMMERCIAL COMPARISON

Plants of ‘Vista2’ are similar in some horticultural characteristics to the variety Bougainvillea ‘Rijnbo710’, PP 21,009. The new variety, however, produces smaller foliage, and flowers bracts with orange coloration, whereas ‘Rijnbo710’ has red-purple bracts. Additionally, inflorescence of ‘Vista2’ are not as tightly compact as inflorescence of ‘Rijnbo710’. Plants of the new variety are more compact than plants ‘Rijnbo710’.

### BRIEF DESCRIPTION OF THE PHOTOGRAPH

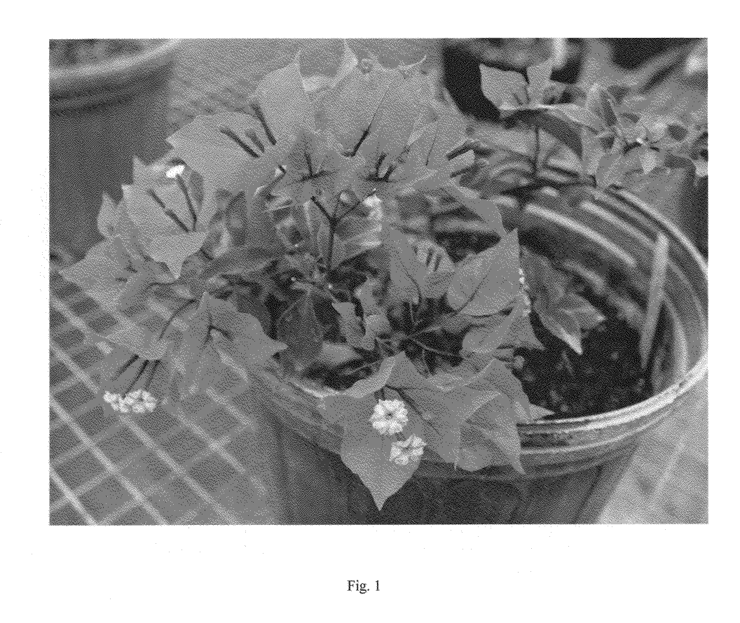

The accompanying photograph in FIG. 1 illustrates in full color a typical plant of ‘Vista2’ grown in a commercial greenhouse in Juana Diaz, Puerto Rico. This plant is approximately 6 months old, shown planted in a 6 inch container. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe a ‘Vista2’ plant. This plant was grown in a greenhouse in a commercial 6 inch pot, in Juana Diaz, Puerto Rico. The plant described is approximately 6 months old. Temperatures ranged from 15° C. at night and 18° C. to 24° C. during the day. No artificial light, photoperiodic treatments were given to the plants. Measurements and numerical values represent averages of typical plant types.
Botanical classification: Bougainvillea communis ‘Vista2’
Propagation:
Time to initiate roots: 14-21 days at approximately 25° C.
Root description: Roots are fine, fibrous and well branched.
Plant:
Growth habit: This plant is an upright tender perennial, suitable for indoor ornamental use, or outdoor garden use in warm climates.
Pot size of plant described: 6 inch
Height: 15 cm.
Plant spread: 20 cm.
Main stem color: Grey-Brown 199B
Main stem texture: Smooth, scaly
Branching characteristics: Not free branching, moderate branching can be achieved with pinching.
Length of primary lateral branches: 7-11 cm.
Quantity of primary lateral branches: 3 per plant
Characteristics of primary lateral branches:
   Diameter.—Approximately. 0.3 cm.
   Color.—Near RHS Grey-Purple 183C.
   Texture.—Dull, scaly.
Internode length: 0.9 cm to −2.0 cm
Foliage:
Leaf:
   Arrangement.—Alternate, simple.
   Quantity.—Approximately 8 per branch.
   Average Length.—Approximately 3.2 cm.
   Average Width.—Approximately. 2.3 cm.
   Shape of blade.—Elliptical.
   Apex.—Acute.
   Base.—Acute.
   Margin.—Entire.
   Texture of top surface.—Smooth.
   Texture of bottom surface.—Smooth.
   Appearance of top surface.—Glossy.
   Appearance of bottom surface.—Matte.
   Color.—Young foliage upper side: Near RHS Greyed-Orange 175A Young foliage under side: Near RHS Greyed-Orange 174A Mature foliage upper side: Near RHS Green 137A Mature foliage under side: Near RHS Green 137A.
   Venation.—Type: Pinnate Venation color upper side: RHS Yellow-Green 144B Venation color under side: RHS Yellow-Green N144B.
   Petiole.—Length: 1.1 cm. Diameter: 0.2 cm. Color: near RHS Yellow-Green N144A Texture: Smooth.
Other Characteristics:
Thorns: One thorn is above each leaf. Thorn is pointed downwards; Young thorns are soft. Old thorns are very sharp and strong.
   Average thorn length.—1.5-2.0 cm.
   Average thorn width at base.—0.2 cm.
   Thorn coloration.—Young thorns near RHS Grey-Brown N199C Old thorns RHS Grey-Brown N199A.
Flower:
Normal flowering period: Continuously from early Spring through Fall.
Inflorescence:
   Form.—Axillary, compact cyme.
   Width of entire inflorescence.—10 cm.
   Height of entire inflorescence.—7 cm.
   Quantity of individual flowers in inflorescence.—Avg. 3 on a 6 month old plant.
Flower longevity on plant: Approximately 10 days
Quantity of Inflorescence per plant: Avg. 4
Persistent or self-cleaning: Self-cleaning
Bud:
   Shape.—Oblanceolate with sharp apex.
   Length.—0.4 cm.
   Diameter.—0.3 cm.
   Color.—Near RHS Red 40B.
Individual true flower:
   Form/type.—Single, tubular flowers growing from the base of a single bract. The flowers have no petals or sepals. The individual true “flower” is small, and each cluster of three flowers is surrounded by three bracts. Sometimes the cluster consists of more flowers but the number of bracts is nearly always 3.
   Diameter.—0.8 cm.
   Depth.—0.25 cm.
   Days lasting on plant.—Approximately one week.
Petals: No petals
Bracts:
   Days lasting on plant.—Approximately 10, self-cleaning.
   Shape.—Deltoid.
   Length.—3.4 cm.
   Width.—2.0 cm.
   Tip.—Acute.
   Base.—Rounded.
   Margin.—Entire.
   Aspect.—Undulated.
   Color.—Under side of immature bract: RHS Orange 28A Upper side of immature bract: RHS Orange N25A Under side of mature bract: RHS Orange N25A Upper side of mature bract: RHS Orange 28A Bract vein color on under side: RHS Orange-Red N34A Bract vein color on upper side: RHS Green 143B.
Sepals: No sepals observed
Peduncles:
   Length.—Average 4.5 cm.
   Diameter.—0.2 cm.
   Angle.—Approximately 70-90° to the lateral branch.
   Strength.—Moderately strong.
   Texture.—Smooth.
   Color.—Near RHS Greyed-Orange 1 73A.
Pedicels:
   Arrangement.—Individual flowers in whorls of 3, together on pedicels. Each individual flower sessile, implanted in a colorful bract.
   Length.—2.2 cm.
   Diameter.—0.1 cm.
   Angle.—Average angle of 45 degrees.
   Strength.—Moderately strong.
   Color.—Near RHS Greyed-Orange 174A.
Fragrance: None
Reproductive organs:
Stamens:
   Number.—Average 8.
Anthers:
   Length.—Approximately 0.05 cm.
   Color.—Near Yellow 7A.
   Pollen.—Color RHS Yellow-Orange 15A Quantity: Scant.
Pistil:
   Number.—1.
   Length.—0.5 cm.
   Style.—Length: 0.1 cm. Color: RHS Yellow-Green 145C.
   Stigma.—Shape: Lineate Color: Near RHS Yellow Green 146C Ovary Color: Near RHS Yellow Green 145B.
Other characteristics:
Seeds and fruits: Seed production not observed.
Disease/pest resistance: Neither resistance nor susceptibility to diseases or pests has been observed in this variety.
Temperature tolerance: Tolerates high temperatures to at least 35 degrees Celsius. Frost is not tolerated. When roots are frozen completely, the plant will not recover.
Drought tolerance: No drought tolerance. Leaves and bracts will drop off the plant immediately under dry conditions.

What is claimed is:

1. A new and distinct cultivar of Bougainvillea plant named ‘Vista2’ as herein illustrated and described.
